(19) United States
(12) Patent Application Publication
(10) Pub. No.: US 2025/0155345 A1
(43) Pub. Date: May 15, 2025

(54) ACOUSTIC FLUID DENSITY DETERMINATION FOR GEOMETRICALLY COMPLEX FLOW CELLS

(71) Applicant: Baker Hughes Oilfield Operations, LLC, HOUSTON, TX (US)

(72) Inventor: Rocco DiFoggio, Houston, TX (US)

(73) Assignee: Baker Hughes Oilfield Operations LLC, HOUSTON, TX (US)

(21) Appl. No.: 18/612,932

(22) Filed: Mar. 21, 2024

Related U.S. Application Data
(60) Provisional application No. 63/599,028, filed on Nov. 15, 2023.
(60) Provisional application No. 63/609,126, filed on Dec. 12, 2023.

(51) Int. Cl. G01N 9/24; E21B 49/10; G01N 33/28

(52) U.S. Cl. CPC G01N 9/24; E21B 49/08; E21B 49/10; G01N 33/2823

(57) ABSTRACT

A method of wellbore operations for identifying sample fluid density and viscosity. The sample fluid is collected from a formation, and acoustically irradiated in a flow cell. Acoustic reflections are collected and analyzed to obtain a sound speed of the sample fluid and a value for the slope of the decay rate of an acoustic signal reverberating within a wall of the flow cell, which is in physical contact with the transducer. The fluid temperature, pressure, and the sound speed of the flow cell material may also be measured. The sample fluid density and viscosity are estimated from equations derived by a regression performed on a set of training data, which was generated from testing the flow cell with fluids having known densities, viscosities, or other characteristics.

### CROSS-REFERENCE TO RELATED APPLICATION

This application claims priority to and the benefit of co-pending U.S. Provisional Application Ser. No. 63/599,028, filed Nov. 15, 2023, and U.S. Provisional Application Ser. No. 63/609,126, filed Dec. 12, 2023, the full disclosures of which are incorporated by reference herein in their entireties and for all purposes.

### BACKGROUND OF THE INVENTION

### 1. Field of Invention

The present disclosure relates to downhole evaluation of fluid sampled from a formation.

### 2. Description of Prior Art

Fluid contained in subterranean formations is sampled and analyzed to identify zones of possible interest in a formation with regard to hydrocarbon bearing potential. This typically involves recovering a sample of any formation fluids present for later analysis in a laboratory environment while causing a minimum of damage to the tested formations. The formation sample is essentially a point test of the possible productivity of subsurface earth formations. Additionally, a continuous record of the control and sequence of events during the test is made at the surface. From this record, valuable formation pressure and permeability data as well as data determinative of fluid compressibility, density and viscosity can be obtained for formation reservoir analysis.

Generally, formation fluid sampling involves disposing a tool into a wellbore equipped with a sample port on its outer housing. Samples are obtained by extending a probe from the tool and pushing that probe up against the wall of the wellbore, then reducing the fluid pressure inside of the probe below formation fluid pressure so that fluid from the formation starts flowing into the tool. The sampled fluid is then analyzed, either within the tool or on surface.

Techniques for analyzing formation fluid with acoustics are provided in DiFoggio, U.S. Pat. No. 7,024,917 (“DiFoggio '917”); DiFoggio et al, U.S. Pat. No. 7,523,640 (“DiFoggio et al. '640”); DiFoggio et al, U.S. Pat. No. 7,614,302 (“DiFoggio et al. '302”); DiFoggio et al, U.S. Pat. No. 7,694,734 (“DiFoggio et al. '734”); DiFoggio et al, U.S. Pat. No. 7,921,691 (“DiFoggio et al. '691”); DiFoggio et al, U.S. Pat. No. 10,570,733 (“DiFoggio et al. '733”); DiFoggio et al, U.S. Pat. No. 11,156,084 (“DiFoggio et al. '084”); and DiFoggio et al, U.S. Pat. No. 11,768,178 (“DiFoggio et al. '178”), which are assigned to the assignee of the present application and incorporated by reference herein in their entireties and for all purposes. For years, downhole fluid density and viscosity were measured in real time using a tiny piezoelectric tuning fork as described in DiFoggio et al. '734. However, as logging jobs have gotten much longer, correspondingly longer-lasting density and viscosity sensors became desired, as explained DiFoggio et al. '178, as well as a backup sensor should the tuning fork fail in the middle of a long logging job, which prompted this renewed interest in an acoustic technique to measure downhole fluid density and viscosity.

### SUMMARY OF THE INVENTION

Disclosed herein is a method of estimating a formation fluid property that includes obtaining sets of data that each have a corresponding sound speed, temperature, pressure, and density value of a live crude oil sample, generating multinomial expansions from the sets of data to form expanded sets of data, developing a correlation prediction function by performing a regression on the sets of data and the expanded sets of data, collecting fluid from the formation to define a sample fluid, obtaining a value of sound speed of the sample fluid by acoustically analyzing the sample fluid inside of an evaluation flow cell, and estimating a density of the sample fluid based on the sound speed from the acoustical analysis and the correlation prediction function. The method further optionally includes obtaining values of a reverberation decay rate by acoustically analyzing the sample fluid inside of an evaluation flow cell. In an example, the fitting equation for x has the form of: x=k₀+Σᵢ (kᵢ ρc^{m} C_{c}ⁿ C_{F}^{p} S^{q} T^{r} P^{v} Log (ρ_{c})^{a} Log (C_{c})^{b} log (C_{F})^{d} log(S)^{f} log (T)^{g} log (P)^{h}), where x is one of fluid density and the logarithm of fluid density and where the exponents can be any real number including zero. In an embodiment predictions of fluid viscosity are made from the calculated fluid density, and alternatively predictions of brine salinity are made from the calculated fluid density. In an example, predictions of multiple fitting equations are generated, a median is taken, which defines the outliers, and predictions, which are not outliers, are averaged. Known density fluids are optionally used to correct the prediction equations to individualize the prediction equations optimized for each individual serialized flow cell using a bias and skew correction. The step of collecting is alternatively performed within a wellbore that intersects the formation, and that further optionally includes conducting wellbore operations in the wellbore based on a value of the sample fluid density. In one example, the step of acoustically analyzing is performed in the wellbore.

Another method of estimating a formation fluid property is disclosed that includes obtaining sets of data that each comprise corresponding values of a sound speed and at least one of a condition or property of a live crude oil sample, generating multinomial expansions from the sets of data to form expanded sets of data, developing a correlation prediction function by performing a regression on the sets of data and the expanded sets of data, collecting fluid from the formation to define a sample fluid, obtaining a value of sound speed of the sample fluid by acoustically analyzing the sample fluid inside of an evaluation flow cell, and estimating a density of the sample fluid based on the sound speed from the acoustical analysis and the correlation prediction function. In an example, the live oil sample condition is temperature or pressure and the live oil sample property is viscosity or density.

### BRIEF DESCRIPTION OF DRAWINGS

Some of the features and benefits of the present invention having been stated, others will become apparent as the description proceeds when taken in conjunction with the accompanying drawings, in which:

FIG. 1 is partial side sectional view of an example of collecting formation fluid with a downhole tool.

FIG. 2 is a side sectional view of an example of a sensor assembly for use with the downhole tool of FIG. 1.

FIG. 3 is an axial sectional view of the sensor assembly of FIG. 2 and taken along lines 3-3.

FIG. 4 is side sectional view of an alternate example of a sensor assembly for use with the downhole tool of FIG. 1.

FIGS. 5-7 are graphs of predicted versus observed fluid density values based on different equations and using the flow cell of FIG. 3.

FIGS. 8a-8c are graphs of predicted versus observed density values for different fluids using the flow cell of FIG. 3.

FIG. 9 is a graph of predicted versus observed density values of downhole crude oils and using the flow cell of FIG. 3.

FIG. 10 is a graph of predicted versus observed viscosity values of downhole crude oils and using the flow cell of FIG. 3.

FIG. 11 is a graph of predicted versus observed viscosity values of brine salinity of formation fluid using the flow cell of FIG. 3.

While subject matter is described in connection with embodiments disclosed herein, it will be understood that the scope of the present disclosure is not limited to any particular embodiment. On the contrary, it is intended to cover all alternatives, modifications, and equivalents thereof.

### DETAILED DESCRIPTION OF INVENTION

The method and system of the present disclosure will now be described more fully hereinafter with reference to the accompanying drawings in which embodiments are shown. The method and system of the present disclosure may be in many different forms and should not be construed as limited to the illustrated embodiments set forth herein; rather, these embodiments are provided so that this disclosure will be thorough and complete, and will fully convey its scope to those skilled in the art. Like numbers refer to like elements throughout. In an embodiment, usage of the term “about” includes +/−5% of a cited magnitude. In an embodiment, the term “substantially” includes +/−5% of a cited magnitude, comparison, or description. In an embodiment, usage of the term “generally” includes +/−10% of a cited magnitude.

It is to be further understood that the scope of the present disclosure is not limited to the exact details of construction, operation, exact materials, or embodiments shown and described, as modifications and equivalents will be apparent to one skilled in the art. In the drawings and specification, there have been disclosed illustrative embodiments and, although specific terms are employed, they are used in a generic and descriptive sense only and not for the purpose of limitation.

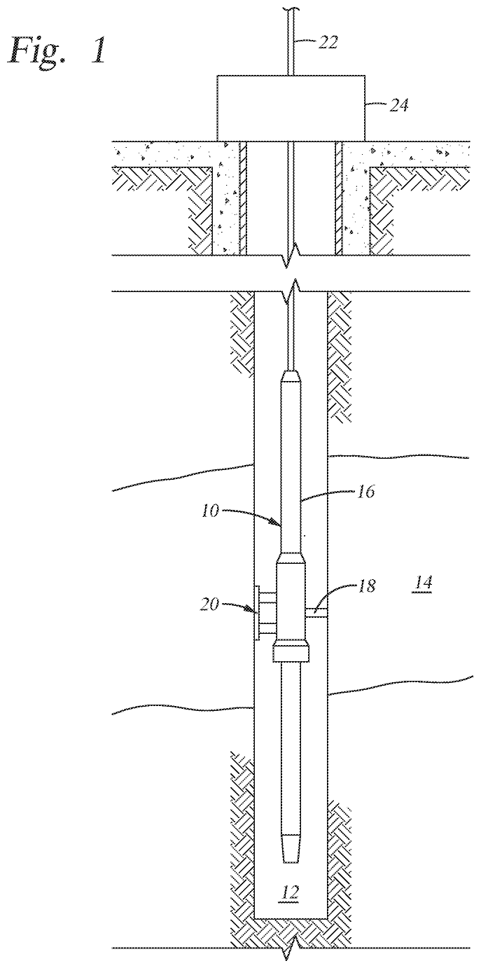

An example of a downhole fluid sampling tool 10 in a wellbore 12 is shown in a side partial sectional view in FIG. 1. Wellbore 12 is formed into a subterranean formation 14, in which fluid is to be sampled with the tool 10. Tool 10 includes an outer housing 16, an annular probe 18 shown extending radially outward from housing 16, and a piston assembly 20 on a side of housing 16 spaced circumferentially away from probe 18. Piston assembly 20 is shown in a deployed configuration and exerting a force against a sidewall of wellbore 12; this in turn urges tool 16 in a radially opposite direction to insert probe 18 into the surrounding formation 14. A bore (not shown) inside probe 18 provides communication between formation 14 and to inside of housing 16. In this example, tool 10 is deployed inside the wellbore 12 on wireline 22, which extends through a wellhead assembly 24 on surface. Optionally, an end of wireline 22 opposite tool 10 connects to a spool on a service truck (not shown) on surface.

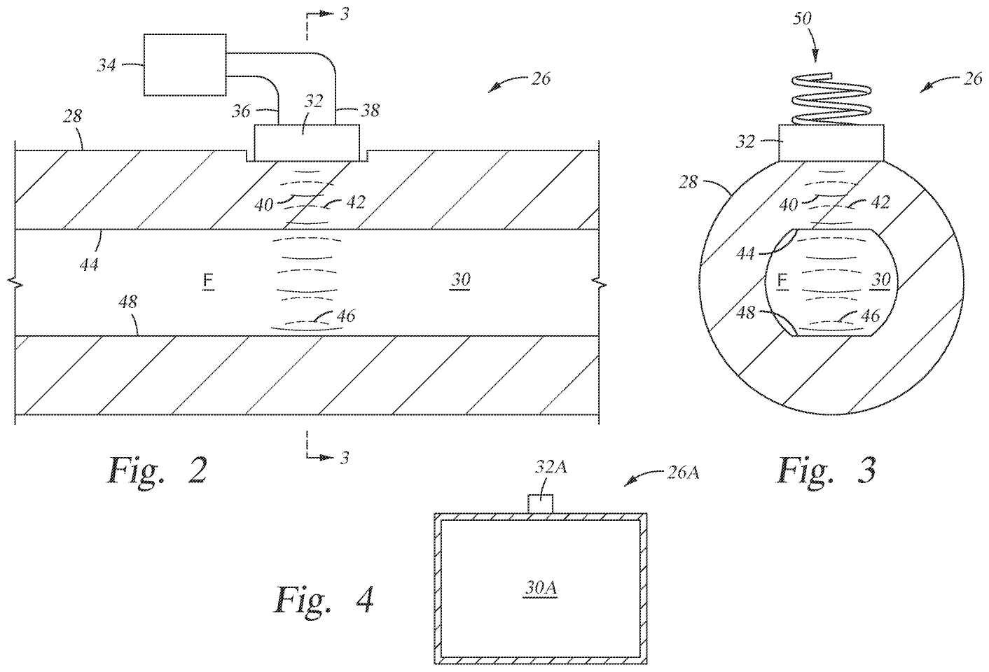

In a side partial sectional view in FIG. 2 is an example of a sensor assembly 26 for analyzing sample fluid F gathered from the formation 14 (FIG. 1). Included with sensor assembly 26 is an annular flow tube 28 having a passage 30 extending axially through the flow tube 28, and a transducer 32 mounted on an outer surface of flow tube 28. In an example, transducer 32 is made of or includes electroactive components such as a piezo-electric materials, electroactive polymers, and combinations thereof. Sensor assembly 26 optionally includes an optional pulse generator 34 and leads 34, 36 connected between the transducer 32 and generator 34. In a non-limiting example of operation, electricity is transmitted to the transducer 32 from the generator 34 through one of the leads 34, 36. Energizing the transducer 32 causes a vibratory response in the transducer 32 so that the transducer 32 operates as a transmitter. When operating as a transmitter, the transducer 32 creates compressional waves 40, which are shown propagating from an interface between the transducer 32 and outer surface of the flow tube 38 into a wall of the flow tube 38. The compressional waves 40 pass radially through the wall and into the passage 30. Reflected waves 42 are generated as the compressional waves 40 cross a wall 44 of passage 30 proximate the transducer 32, and reflected waves 46 are generated as the compressional waves 40 cross a wall 48 of passage 30 distal from the transducer 32. The reflected waves 42, 46 are directed towards the transducer 32. In examples the transducer 32 also operates as a receiver, and in examples, is responsive when waves 42, 46 arrive at the transducer 32. In an embodiment of a response of transducer 32 to the arrival of waves 42, 46, signals are generated by transducer 32, which in examples are transmitted to generator 34 via one or more of leads 36, 38. Examples of such signals are disclosed in DiFoggio '917, DiFoggio et al. '640, DiFoggio et al. '302, and DiFoggio et al. '691. In the example of FIG. 2, sample fluid F is flowing through the passage 30, in alternatives sample fluid F is static inside passage 30.

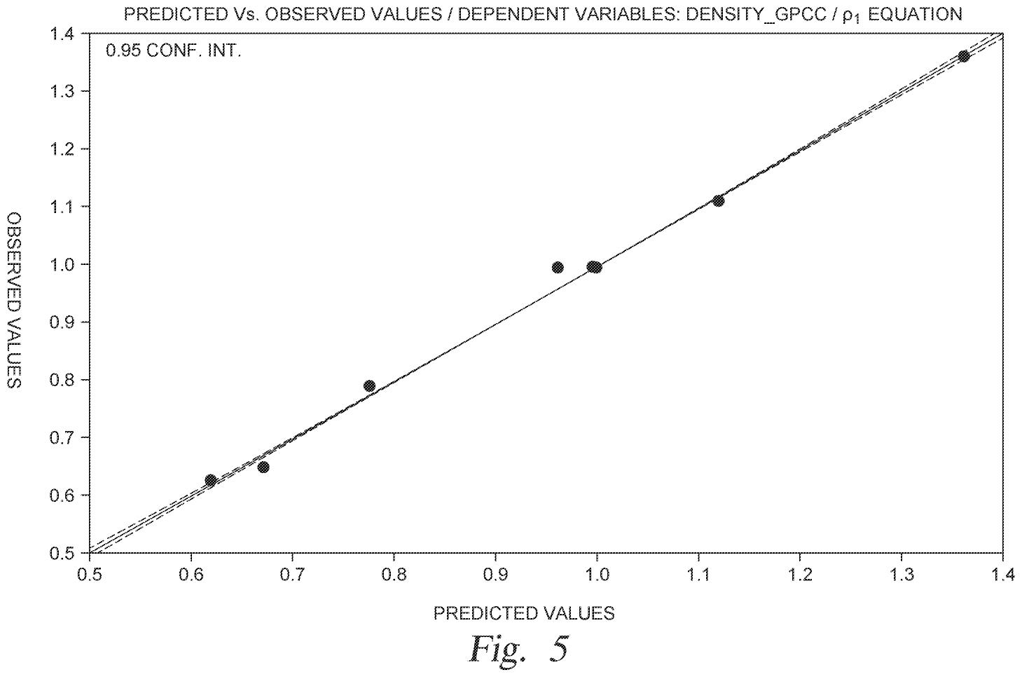

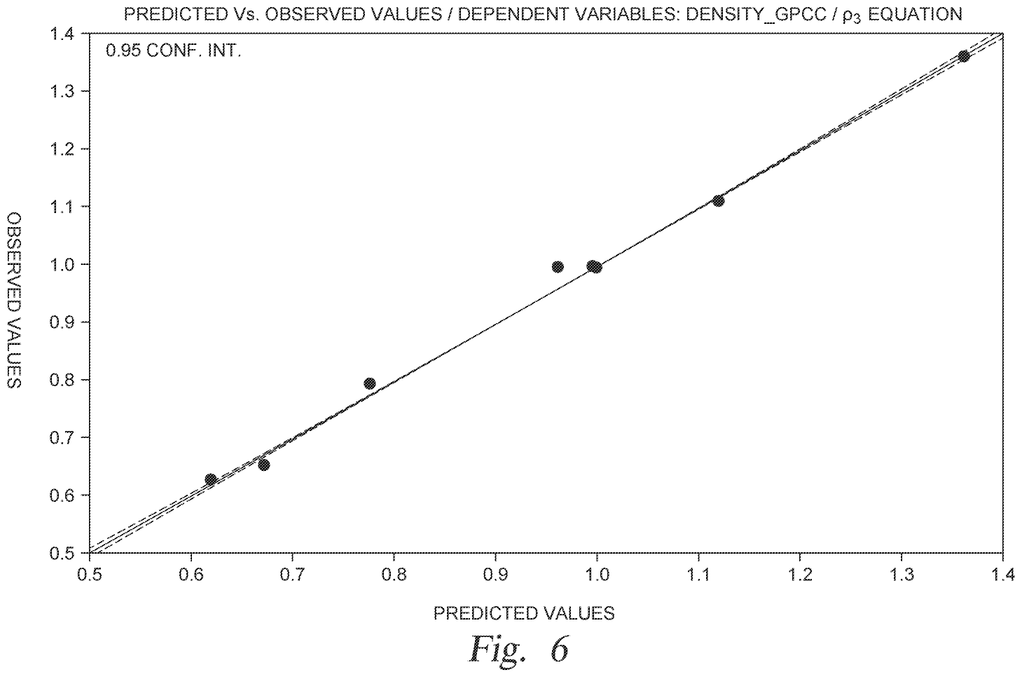

Referring now to FIG. 3, examples of the sensor assembly 26 include a biasing means 50 (such as a spring) for urging the transducer 32 against the outer surface of the flow tube 28 and to maintain contact between the transducer 32 and flow tube 28. Optionally, a high temperature acoustic coupling grease (not shown) is provided on the interface between the transducer 32 and flow tube 28. As shown in the axial view of FIG. 3 is that the shape of the walls 44, 48 proximate and distal to transducer 32 are planar and portions of sidewall between walls 44, 48 are curved. Because of the particular shape of the flow cell cross section of FIG. 3, and the large size of the transducer compared to the flow cell, it is not amenable to a simple theoretical model. However, there are existing downhole tools with flow cells for sound speed with this unusual shape. Replacing the flow cells of these tools is impractical because of serious size constraints within the existing tools as well as budget constraints. Therefore, a need exists to find empirical equations to convert acoustic waveforms collected by these existing tools to a fluid density, which is an advantage provided by the present disclosure. To account for slight manufacturing differences in cell pathlengths, each individual flow cell is serialized and calibrated for sound speed as a function of pressure in pure water against a 1 ppm oceanographic sound speed reference sensor. Serialization also permits any modeling equations that are developed to be optimized to each individual, serialized flow cell, perhaps, with a bias and skew adjustment. In FIG. 4, the cross-section plane is perpendicular to the flow tube axis. Referring now to FIG. 4, shown is an alternate example of a flow cell 26A having a passage 30A with a cross-section that is rectangular and is much larger than the transducer 32A, which results in the theoretical analysis being straightforward. Then, it can be assumed that the plane compressional wave created by the transducer propagates through the flow cell wall and internal fluid and returns largely as a plane wave with minimal interference from shear wave or Stonely wave conversions at the interfaces. Also, the following can be calculated: ρ_{Theory}=(ρ_{flowcell}*c_{flowcell}/C_{fluid})*[(1−sqrt (F)] [(1-sqrt (F)]: where F=exp (m_{fluid}−mₐᵢᵣ). Here, m_{fluid} is the initial slope of the natural log of the reverberation ringdown within the flow cell wall that is in contact with transducer when a fluid, such as crude oil, is in the flow cell and m air is the initial slope of this reverberation when air is in the flow cell. The known thickness of the wall divided by the time spacing between these within wall reverberations yields the sound speed c flow cell of the flow cell material.

In a non-limiting example of operation, sample fluid F obtained from the formation 14 (FIG. 1) flows through passage 30 inside flow tube 28 while the waves 40 generated by transducer 32 produce reflected waves 42, 46 that are sensed by transducer 32. Flow tube 28 is optionally referred to herein as a flow cell. Signals are generated by transducer 32 indicating an energy magnitude and time arrival of reflected waves 42, 46. Embodiments exist in which these signals are the same or similar to those disclosed in one or more of DiFoggio '917, DiFoggio et al. '640, DiFoggio et al. '302, and DiFoggio et al. '691. Optionally, electricity transmitted to the transducer 32 from the generator 34 is pulsed for a time period that ends before waves 42, 46 arrive at the transducer 32. Similar to that described in DiFoggio et al. '640 and DiFoggio et al. '733, the signals used identify the sample fluid F sound speed (“C_{F}”) and a slope (“S”) of reverberation peaks of waves 42, 46. With knowledge of the flow cell sound speed (“C_{c}”) from a table or by measurement, and flow cell material density (“ρ_{c}”) from a table, sample fluid F density (“ρ_{F}”) is estimated based on results from equations (1)-(3) below:

$${ρ}_{F} = - 0.811572399 + ( 0.000748061 ) ( {C}_{F} ) + ( 0.356716474 ) ( {S}^{2} ) - ( 0.311779206 ) ( {S}^{- 1} ) \qquad ( 1 )$$

$${ρ}_{F} = 2.023881 - ( 0.053727 ) ( {ρ}_{c} ) ( {C}_{c} ) ( {C}_{F}^{- 1} ) \qquad ( 2 )$$

$${ρ}_{F} = 0.191295 + ( 0.000772 ) ( {C}_{F} ) + ( 0.61373 ) ( {S}^{2} ) + ( 0.954792 ) ( S ) \qquad ( 3 )$$

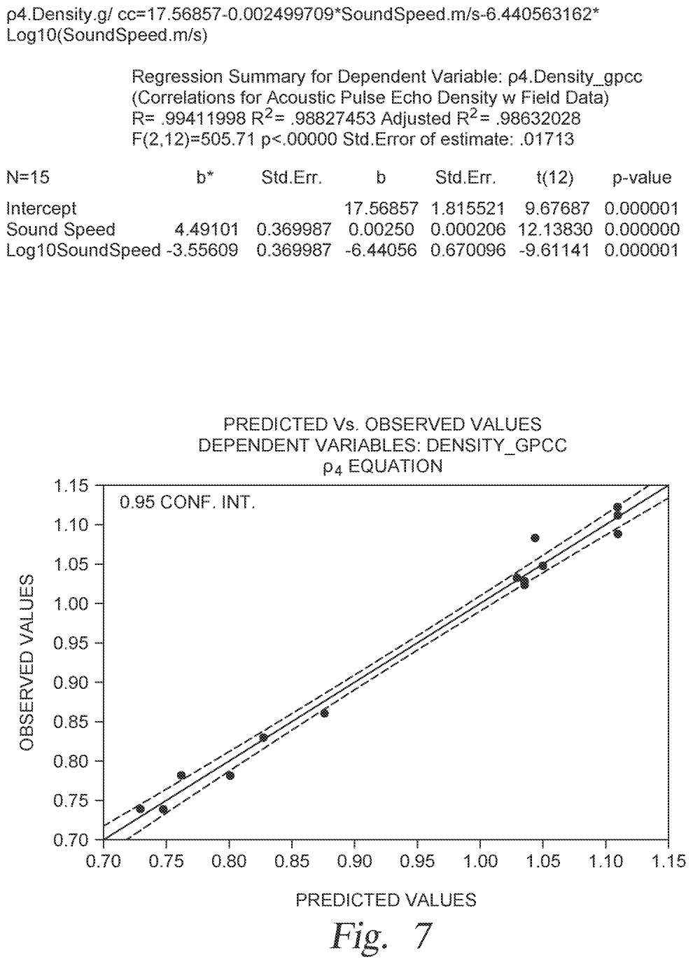

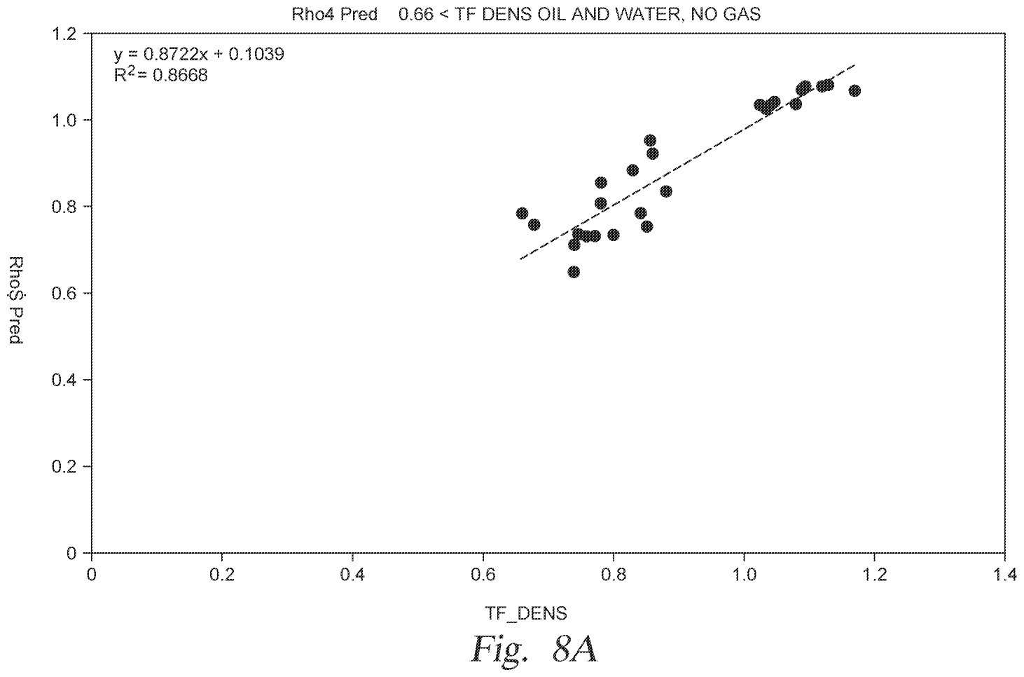

The process for deriving Equations (1)-(3) included directing different fluids having known densities through a sensor assembly with a flow tube (or flow cell) having the same configuration of flow tube 28 (i.e., planar opposing inner walls), energizing the transducer 32, and collecting data based on responsive signals from the transducer 32 as explained above. The different fluids included pentane, hexane, decane, methanol, water, ethylene glycol, and honey, with 9 to 20 repeats of each sample. Equations (1)-(3) are empirical equations derived from an all-combination search using step-forward multiple linear regression with substitution, applied to a training set of acoustic data from these different fluids using the STATISTICA® Version 13.5.0.17 analytics software package (TIBCO Software Inc., USA). Examples of how these empirical equations are derived are found in DiFoggio et al. '733 and DiFoggio et al. '084. Initially, it was uncertain whether this purely empirical approach with no theoretical basis would work at all. However, it was a complete surprise that it was possible to achieve R² values in excess of 0.99 for the very simple Equations (1) and (3) as shown in FIGS. 5 and 6, which are predicted versus observed fits to acoustic data given by different equations for a training set that was taken using the flow cell of FIG. 3 for multiple acoustic waveforms of pentane, hexane, decane, methanol, water, ethylene glycol, and honey at room temperature and pressure. An R² of 0.99 means that 99% of the variance has been explained. An R² of 1.00 is perfection. Currently, each sound speed flow cell is given a serial number and is individually calibrated with water against a 1 part per million commercial sound speed sensor at a series of high pressures to account for slight differences each flow cell due to normal manufacturing tolerances as well as for slight stretching of the flow cell at very high internal pressure. It is quite possible that predictions made by Equations (1)-(3) for data collected by flow cells of different serial numbers will lie on a straight line, which is not the equal value line, y=x, but which has an offset (bias) and a tilt (skew) away from the equal value line and is y=mx+b. This can be corrected by first subtracting b from each prediction and then dividing by m to get a corrected prediction, (y-b)/m. In alternatives, other unusual shaped flow cells are similarly modeled to yield fluid density using equations having the form of a constant plus a sum of other constants times products or ratios of one or more of the same four acoustic parameters or the squares of these parameters as shown algebraically in the equation below where k₁ are fitting constants determined by the regression process and are unique to each flow cell shape and m, n, p, and q can each be any of 2, 1, 0 or −1. Note that, if there is only a single parameter in a term of the fitting equation, then the powers of the other parameters in that four-parameter product of Equation 4 below are all equal to zero.

$${ρ}_{F} = {k}_{0} + {Σ}_{i} ( {k}_{i} {ρ}_{c}^{m} {C}_{c}^{n} {C}_{F}^{p} {S}^{q} ) \qquad ( 4 )$$

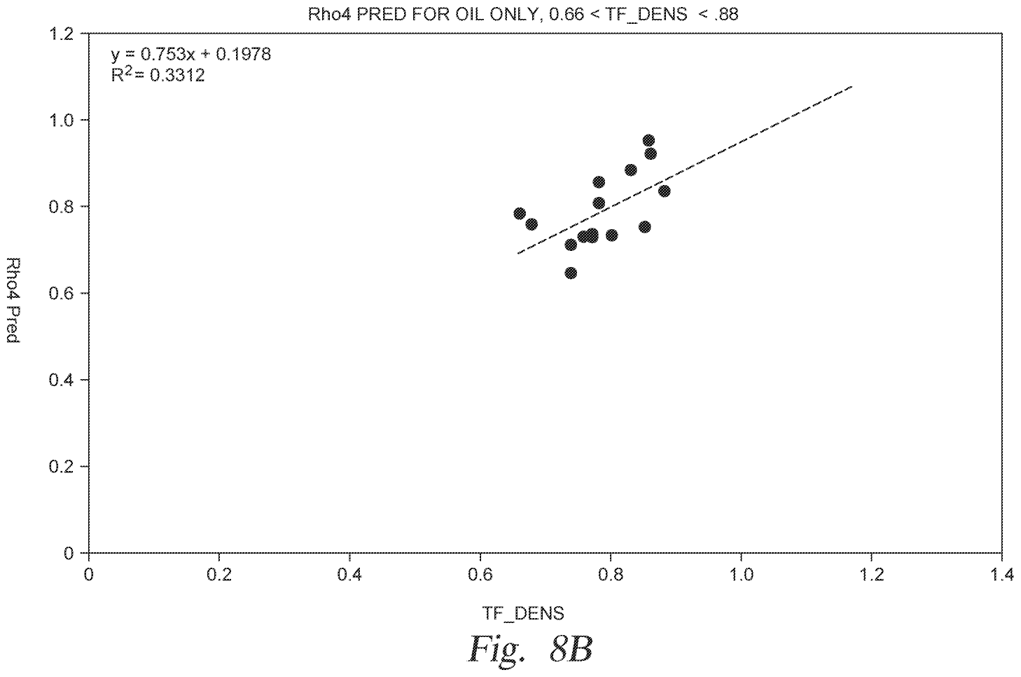

It was found that these first three equations, which were based upon modeling of acoustic waveforms of pure laboratory samples at room temperature and pressure, did not predict as accurately for real downhole fluids at reservoir temperatures and pressures. Therefore, a new empirical model, ρ₄, was developed, which was based upon both lab samples at room temperature and pressure and field data of downhole fluids at reservoir conditions and this equation used only fluid sound speed, C_{F}, as input as shown in FIG. 7, which is the ρ₄ model for fluid density whose R² is 0.99 with a standard error of calibration of 0.017 g/cc. It is based upon both the pure fluid lab samples of FIGS. 5 and 6 (at room temperature and pressure) and field data of downhole fluids (at reservoir temperatures and pressures) and it uses only sound speed as input. A ρ₅ model based only upon downhole fluid samples using sound speed and logarithm of sound speed had almost identical fitting parameters and predictions so it is not repeated here.

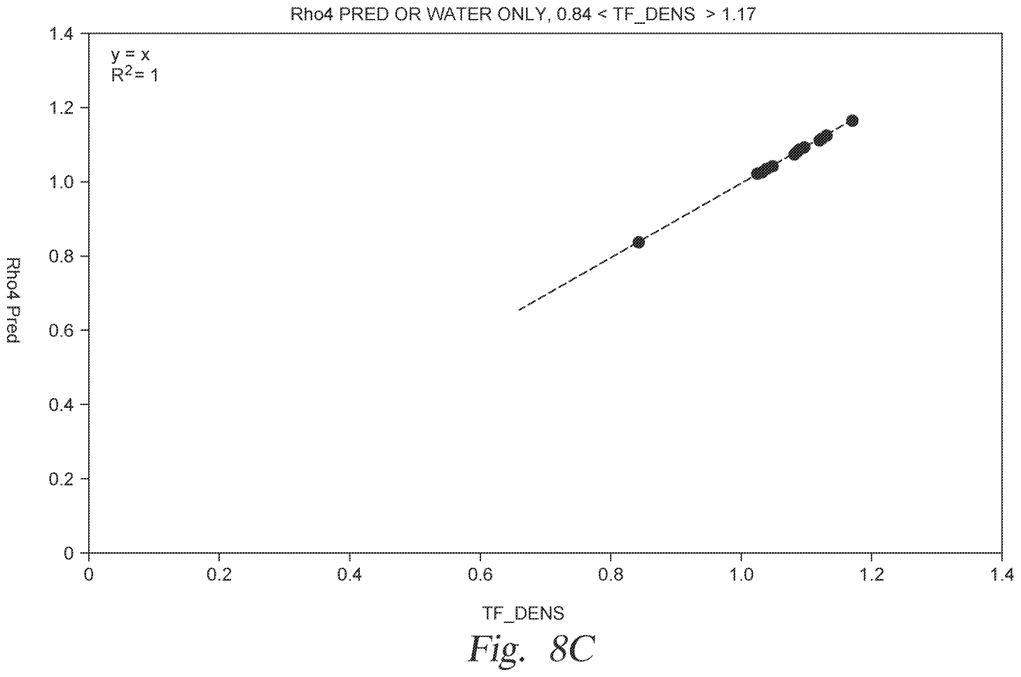

FIGS. 8a-8c show that this ρ₄ model had a perfect, R² of 1.00, for predicting the densities of downhole brines but it was not as good at predicting the densities of downhole crude oils, which is commercially more important. One reason might be because it did not explicitly include temperature or pressure in the model.

Equation 4 for the form of the modeling equation is now revised to Equation 5 to explicitly include (T) temperature and (P) pressure. It has the form of an offset constant, k₀, plus a sum over all kᵢ of the product of kᵢ with twelve parameters, each raised to an exponent. These exponents m, n, n, p, q, r, and v, a, b, d, g, f are now generalized to all real numbers, including zero. A zero exponent makes that factor in the product equal to one and thereby removes any dependence upon that variable. In Equation 5, the variable x is one of the fluid density and the logarithm of the fluid density.

$$x = {k}_{0} + {Σ}_{i} ( {k}_{i} {ρ}_{c}^{m} {C}_{c}^{n} {C}_{F}^{p} {S}^{q} {T}^{r} {P}^{v} log {( {ρ}_{C} )}^{a} log {( {C}_{C} )}^{b} log {( {C}_{F} )}^{d} log {( S )}^{f} log {( T )}^{g} log {( P )}^{h} ) \qquad ( 5 )$$

In the terminology of Equation 5, for ρ₄, k₀=17.56857, with k₁=−0.002499709 multiplying the fluid sound speed, C_{F}, raised to the exponent q=1, and k₂=−6.440563162 multiplying the logarithm of the fluid sound speed, log (C_{F}) raised to the exponent f=1 and all other kᵢ and all other exponents are equal to zero, which then simplifies to Equation 6.

$${ρ}_{4} = 17.56 \; 857 - 0.002499709 {C}_{F 1} - {6.440563162 [ log ( {C}_{F} ) ]}_{1} \qquad ( 6 )$$

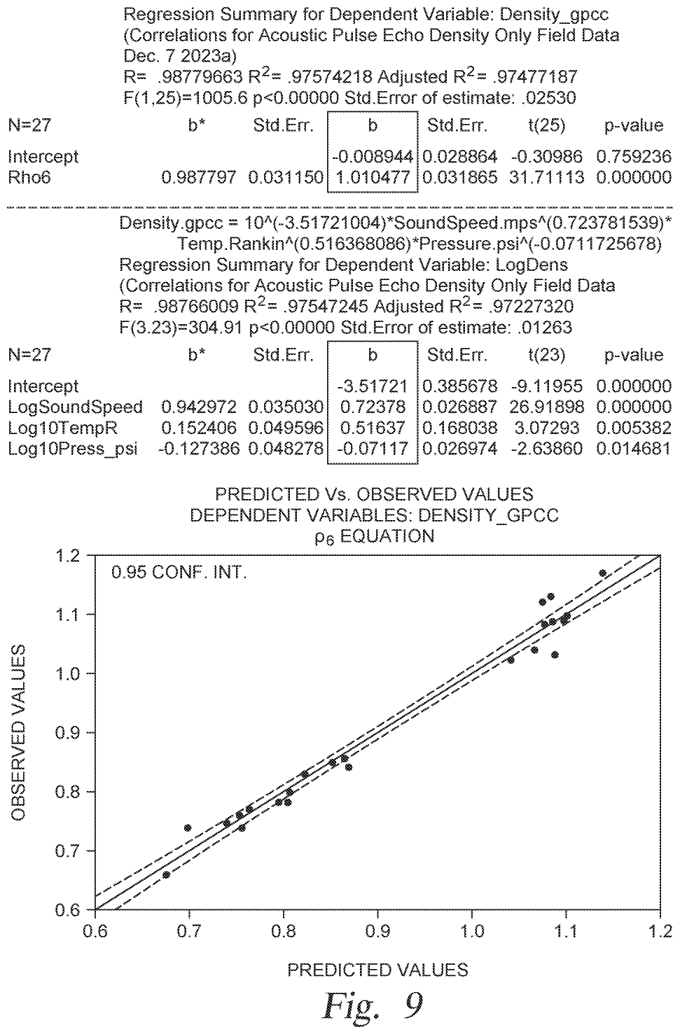

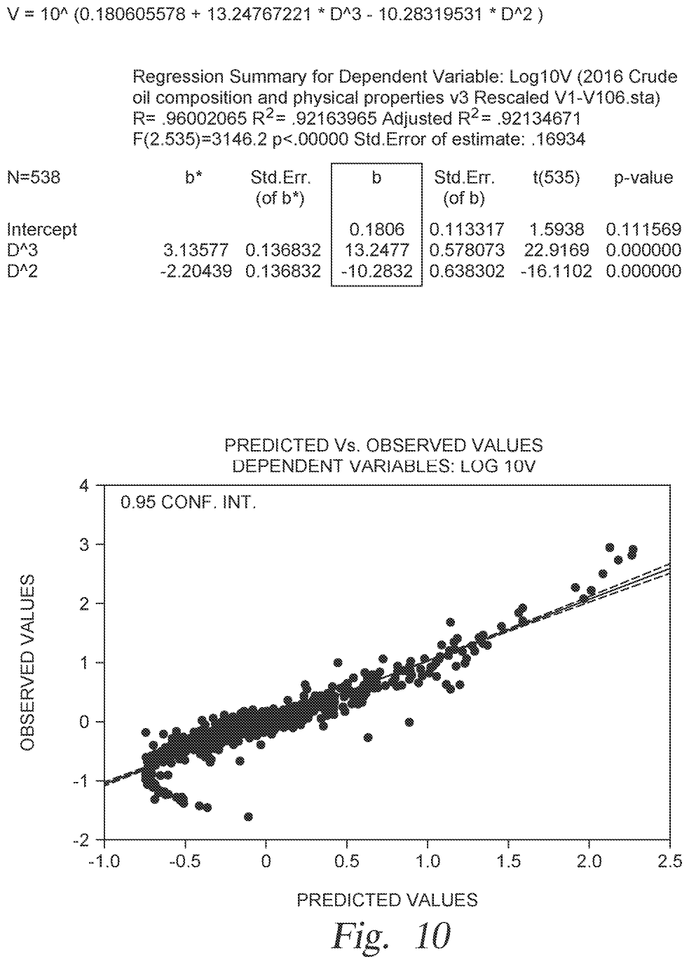

A new empirical model, ρ₆, shown in Equation 7 was developed, which is based only upon downhole crude oils at reservoir conditions, and as shown in FIG. 9 this equation explicitly includes temperature and pressure along with sound speed. An equation, also referred to herein as a correlation prediction function (“correlation”) was developed to estimate live crude oil viscosity directly from live crude oil density, which is based upon data sets made up of corresponding conditions and properties of 538 live crude oil samples at reservoir temperatures and pressures as shown in FIG. 10. This correlation has a one sigma logarithmic uncertainty of 0.17, which corresponds to an uncertainty factor of 10 (0.17)=1.48. That is, 68.27% of the time a 1 cP fluid's viscosity will be estimated between (1 divided by 1.48) cP=0.68 cP and (1 multiplied by 1.48) cP=1.48 cP This correlation is best when the logarithm of viscosity exceeds −0.8, which corresponds to viscosity exceeding 0.16 cP. In the terminology of Equation 5, for ρ₆, it is estimated that the logarithm of density as k₀=17.56857 plus k₁=−0.002499709 times the logarithm of fluid sound speed raised to the exponent d=1 plus k₂=−6.440563162 times the logarithm of the Rankine temperature raised to the exponent g=1 plus k₃=−0.0711726 times the logarithm of the pressure in psi raised to the exponent h=1 with all other kᵢ and all other exponents are equal to zero, which then simplifies to Equation 7. Fluid viscosity can be estimated from a calculated fluid density as shown in FIG. 10 and Equation 8.

$${Log}_{10} ( {ρ}_{6} ) = - 3.5172100 + 0.7 \; 237815 {Log}_{10} ( {C}_{F} ) + 0.5163681 {Log}_{10} ( T ) - 0.071 \; 1726 {log}_{10} ( P ) \qquad ( 7 )$$

$${Log}_{10} ( Viscosity ( cP ) ) = 0.1806 - 10.2832 {ρ}_{F}^{2} + 13.2477 {ρ}_{F}^{3} \qquad ( 8 )$$

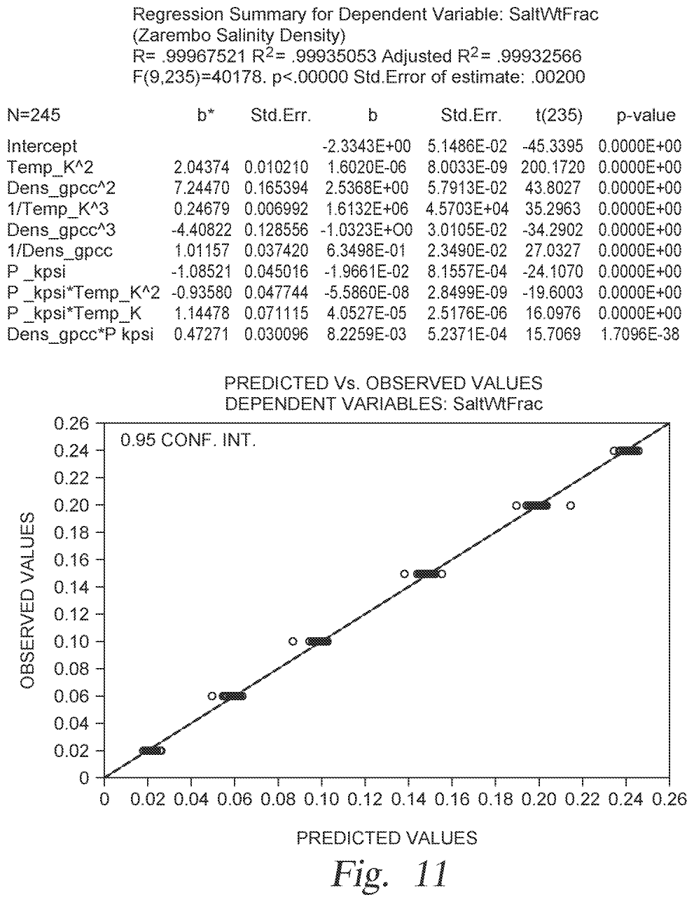

Sodium chloride brine salinity can be estimated from density, pressure, and temperature based upon a newly developed Baker Hughes fitting equation (FIG. 11) for the brine data published in 1975 (Zarembo, V. I., and Fedorov, M. K., 1975, Density of sodium chloride solutions in the temperature range 25-350° C. and at pressures up to 1000 kg/cm²: J. Appl. Chem. USSR, 48, 1949-1953, English translation in 1976). In FIG. 11, the fitting coefficients appear under the column heading, B, so that the fitting equation is the intercept, −2.3343+1.6020 E-06*T² (° K) and so on. Real oil field brines contain mostly sodium chloride, but they can also have smaller amounts of calcium, magnesium, potassium or other cations and smaller amounts of bromine, iodine, or other anions that make up the total dissolved solids in the brine.

In embodiments of the present disclosure, a calculated value of sample fluid density may be established when two or more of Equations (1)-(7) agree. Optionally, when two agree, the third value is discarded and the two agreeing values are averaged. Optionally, more than three fitting equations can be compared, a median value taken, outliers of high leverage discarded, and the predictions, which are not outliers, can be averaged. In another alternative, operations within wellbore 12 (FIG. 1) are changed or undertaken based on a calculated value of sample fluid density; such as a decision to perforate and produce from one or more particular zones in the formation 14, drill a lateral well, conduct fracturing, block production from a lateral well or a particular depth, or some other production or exploration function where a decision to produce or explore is based on knowledge of a composition of a fluid being analyzed as disclosed herein.

The present invention described herein, therefore, is well adapted to carry out the objects and attain the ends and advantages mentioned, as well as others inherent therein. While a presently preferred embodiment of the invention has been given for purposes of disclosure, numerous changes exist in the details of procedures for accomplishing the desired results. These and other similar modifications will readily suggest themselves to those skilled in the art, and are intended to be encompassed within the spirit of the present invention disclosed herein and the scope of the appended claims.

What is claimed is:

1. A method of estimating a formation fluid property comprising:
obtaining training sets of data that each comprise a corresponding sound speed, temperature, pressure, and density value of a live crude oil sample;
generating multinomial expansions from the training sets of data to form expanded sets of data;
developing a correlation prediction function by performing a regression on the training sets of data and the expanded sets of data;
collecting fluid from the formation to define a sample fluid;
obtaining a value of sound speed of the sample fluid by acoustically analyzing the sample fluid inside of an evaluation flow cell; and
estimating a density of the sample fluid based on the sound speed from the acoustical analysis and the correlation prediction function.

2. The method of claim 1, further comprising obtaining values of a reverberation decay rate by acoustically analyzing the sample fluid inside of an evaluation flow cell.

3. The method of claim 1, wherein the correlation prediction function for x has the form of:
x=k₀+Σᵢ(kᵢρ_{C}^{m}C_{C}ⁿC_{F}^{p}S^{q}T^{r}P^{v} log(ρ_{C})^{a} log(C_{c})^{b} log(C_{F})^{d} log(S)^{f} log(T)^{g} log(P)^{h}),
where x is one of fluid density and the logarithm of fluid density and where the exponents can be any real number including zero.

4. The method of claim 1, wherein predictions of fluid viscosity are made from the calculated fluid density.

5. The method of claim 1, wherein predictions of brine salinity are made from the calculated fluid density.

6. The method of claim 1, wherein predictions of multiple fitting equations are generated, a median is taken, which defines the outliers, and predictions, which are not outliers are averaged.

7. The method of claim 1, wherein known density fluids are used to correct the correlation prediction functions to individualize the correlation prediction functions optimized for each individual serialized flow cell using a bias and skew correction.

8. The method of claim 1, wherein the step of collecting is performed within a wellbore that intersects the formation.

9. The method of claim 7, further comprising conducting wellbore operations in the wellbore based on a value of the sample fluid density.

10. The method of claim 5, wherein the step of acoustically analyzing is performed in the wellbore.

11. A method of estimating a formation fluid property comprising:
obtaining training sets of data that each comprise corresponding values of a sound speed and at least one of a condition or property of a live crude oil sample;
generating multinomial expansions from the sets of data to form expanded sets of data;
developing a correlation prediction function by performing a regression on the sets of data and the expanded sets of data;
collecting fluid from the formation to define a sample fluid;
obtaining a value of sound speed of the sample fluid by acoustically analyzing the sample fluid inside of an evaluation flow cell; and
estimating a density of the sample fluid based on the sound speed from the acoustical analysis and the correlation prediction function.

12. The method of claim 11, wherein the live crude oil sample condition is selected from the group consisting of temperature and pressure and wherein the live crude oil sample property is selected from the group consisting of viscosity and density.
